(19) United States
(12) Patent Application Publication
(10) Pub. No.: US 2009/0123954 A1
(43) Pub. Date: May 14, 2009

(54) MULTICOLOR BIOLUMINESCENT VISUALIZATION PROBE SET, OR SINGLE-MOLECULE-FORMAT MULTICOLOR BIOLUMINESCENT VISUALIZATION PROBE

(75) Inventors: Sung Bae KIM, Tsukuba-shi (JP); Hiroaki TAO, Tsukuba-shi (JP); Yoshio UMEZAWA, Saitama-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: National Institute of Advanced Industrial Science and Technology, Tokyo (JP)

(21) Appl. No.: 12/025,532

(22) Filed: Feb. 4, 2008

(30) Foreign Application Priority Data
Aug. 2, 2007 (JP) 2007-202308

(51) Int. Cl. C12Q 1/66; C12Q 1/48; C07H 21/00; C12N 15/63; C12N 5/00

(52) U.S. Cl. 435/8; 435/15; 536/24.3; 435/320.1; 435/325

(57) ABSTRACT

The present invention provides ligand detection means capable of exhibiting two-dimensional information (wavelength and intensity of luminescent signal) responding to multiple signals triggered by a ligand via a target protein, while taking advantage of the merit of the single-molecule-format bioluminescent probe.

The present invention could provide a luminescent probe set which is comprised of a fusion protein comprising a ligand recognition protein and a molecular recognition domain to which the ligand recognition protein bind upon conformational change, wherein the fusion protein is sandwiched between split Lighting Enzyme fragments, the probe set can emit multiple luminescence by utilizing Lighting Enzymes emitting lights with multiple wavelength, wherein these multiple components can be tandemly arranged by using C-terminal fragment of the Lighting Enzyme.

By using a living cell line transfected with gene of multicolor luminescent probe set or single-molecule-format multicolor bioluminescent probe, it becomes possible to distinguish and detect bioactivity level of a target ligand in a complex context of the living cell two-dimensionally (wavelength versus intensity) in multi colors, and to quantitatively evaluate multiple effects (anticancer and carcinogenesis actions, agonist and antagonist) of a ligand represented by a drug at once by two-dimensional information of different colors in short time.

### TECHNICAL FIELD

The present invention relates to a set of multicolor bioluminescent probes or a single-molecule-format bioluminescent probe capable of quickly visualizing two distinctive activities of a ligand that is specific to a target protein.

### BACKGROUND ART

In the natural world, abundant nature and organisms coexist, and maintain their life phenomena based on common molecular mechanisms and principles such as DNA expression. Many researches in medical, pharmaceutical and natural scientific fields aim at finding out life phenomena of living organisms. To understand such life phenomena and ensure safe life environments, it is essential to develop effective novel pharmaceuticals while excluding potential risk factors. For achieving this, it is necessary to biologically analyze various molecular phenomena occurring in a cell line which are the basic units of living organisms (for example, protein-protein interaction, phosphorylation, molecular trafficking, transcription/translation and the like). However, inside cells, (i) uncountable numbers of proteins are present; (ii) internal structures thereof are partitioned into such as organelles and complicated; and (iii) the molecular mechanisms are precisely regulated by unknown complicated and sophisticated control systems. In addition, (iv) detection of target proteins/lipids, which play key roles in life phenomena, is very difficult because they have no specific absorption spectra and generally exist in a small amount in the cells.

Therefore, molecular imaging of specific life phenomena in cell lines or in animal individuals is a main trend in recent researches in wide range of life science fields (Massoud, T. F., and Gambhir, S. S. (2003). Genes & Development 17, 545-580.). In particular, (i) bioanalysis utilizing fluorescent proteins such as green fluorescent proteins (GFPs), and (ii) biological imaging utilizing bioluminescent proteins (particularly, Lighting Enzymes (LEs)) such as firefly luciferases (FLuc) are in the highly competitive research and development fields.

Examples of conventional base techniques using above-described fluorescence and luminescence are (i) FRET (fluorescent resonance energy transfer) method, (ii) BRET (bioluminescence resonance energy transfer) method, (iii) reporter gene assay, (iv) protein complementation, (v) protein splicing and the like. Since these known methods have advantages and disadvantages, they are used in compensative manners. For example, the FRET method and the reporter gene assay which utilize fluorescence as analytical signals have a problem of increase in autofluorescence (background) and hence it requires expensive facility such as sophisticated filtering system for detection of fluorescent signals. They also require an external light source, which makes an in vivo analysis very difficult. On the other hand, the protein complementation and the protein splicing which use luminescence as analytical signals have such a problem of the necessity to introduce external substrates (Kim, S. B.; Ozawa, T.; Watanabe, S.; Umezawa, Y. (2004). Proc. Natl. Acad. Sci. U.S.A. 101, 11542-11547.).

In recent years, the main trends of research in the present field are (i) finding of effective fluorescent/luminescent proteins which are applicable to the above fluorescence/luminescence method, (ii) improvement and optimization for expression of the photoprotein genes in animal cells, and (iii) development of novel bioanalytical method using fluorescence/luminescence. Photoproteins that have been found heretofore (particularly, means “Lighting Enzyme” (LE)) can be generally classified into (i) pH-sensitive LEs and (ii) pH-insensitive LEs. Examples of the pH-sensitive LEs include firefly luciferases (FLucs), Renilla luciferases (RLucs) and the like, and examples of the pH-insensitive LEs include click beetle luciferases (CBLuc), railroad luciferases (RRLucs) and the like (Viviani, V. R., Uchida, A., Viviani, W., and Ohmiya, Y. (2002). Photochem. Photobiol. 76, 538-544.). The aforementioned pH-insensitive LEs are insensitive to heavy metal and temperature, and have characteristics of emitting stable light signals with long wavelengths that are suitable for bioanalysis. For taking advantages of such CBLucs for bioluminescent probes, green luminescent CBLucs and red luminescent CBLucs are employed as LE in the present invention.

Estrogen receptors (ERs) used in examples of the present invention are members of nuclear receptor superfamily, and one of important proteins that are involved in reproduction, growth, metabolism and the like in females. ERs are widely distributed in cells. ERs have such characteristics that upon binding estrogens, they change conformation to form dimers, and after recruiting coactivators or the like, they recognize response element on target gene promoters in the nucleus and bind thereto, to activate transcription of various genes (Zhao, C. Q., Koide, A., Abrams, J., Deighton-Collins, S., Martinez, A., Schwartz, J. A., Koide, S., and Skafar, D. F. (2003). J. Biol. Chem. 278, 27278-27286.).

In various genomic and nongenomic actions of NRs, the conformation of ligand binding domains (LBDs) in NRs differ according to the various properties of ligands, and protein phosphorylation sometimes occurs in LBDs. When ligands having genomic activities bind NRs, NRs acquire binding abilities to coactivators or the like, thereby leading, for example, improvement of intranuclear transcription activities. Such an intramolecular conformational change and dimerization by intermolecular binding are steroid hormone responding mechanisms generally observed in various NRs such as androgen receptors (ARs), progesterone receptors (PRs), glucocorticoid receptors (GRs) and the like as well as in ERs (Kim, S. B., Awais, M., Sato, M., Umezawa, Y., and Tao, H. (2007). Anal. Chem. 79, 1874-1880.). On the other hand, NRs to which ligands having nongenomic activities bind will bind membrane-anchoring proteins represented by kinase, to activate cytoplasmic signal transduction.

The present inventors have previously proposed a probe capable of visualizing binding of cGMP to its target molecule (cGMP-binding protein) based on FRET (fluorescence resonance energy transfer) phenomenon (Japanese Patent Application Laid-open No. 2002-01735), and a probe capable of visualizing IP3 utilizing FRET phenomenon (Pamphlet of International Publication WO2005/113792). Also, the inventors have created a probe, wherein one chromophore is dissected into two reporter molecules (such as luciferases and fluorescent proteins), and the N- and C-terminal sides of the reporter molecules are connected to two independent proteins respecticely, which is not using FRET phenomenon based on energy transfer between two chromophores as an index. Also, the inventors proposed means for detecting protein-protein interactions by using luminescence/fluorescence intensity as index, which is emitted by the spliced or complemented reporter molecules upon binding of the independent two proteins (two-molecular-format probe: Pamphlet of International Publication WO2002/08766 and WO2004/104222). Recently the inventors applied for a patent for a method of visualizing protein-protein interactions in a single molecule based on the principle of self-complementation of LE (Japanese Patent Application No. 2007-005144). The feature of this measure is a method for detecting conformational change of a target protein by one-dimensional-luminescence intensity, wherein the conformational change is induced by binding of a ligand to a target ligand recognition protein which is sandwiched between the N- and C-terminal side fragments of Fluc, pH-sensitive LE.

The aforementioned probe for detecting a target ligand using the FRET phenomenon (Pamphlet of International Publication WO2005/078119, Schaufele, F.; Carbonell, X.; Guerbadot, M.; Borngraeber, S.; Chapman, M. S.; Ma, A. A.; Miner, J. N.; Diamond, M. I. (2005). Proc. Natl. Acad. Sci. U.S.A. 102, 9802-9807), as described above, enables rapid detection of a ligand and it also enables an agonist and antagonist to be detected individually; however, background luminescence is disadvantageously high, and high-resolution fluorescence microscope and sophisticated filter apparatus, as well as an expert technician are required for measuring energy transfer between two chromophores at high accuracy. Additionally, it requires external light source emitting short-wavelength light; therefore bioanalysis is difficult at a level of living subjects which highly absorbs short-wavelength light.

On the other hand, a method of detecting protein-protein interaction utilizing conventional reassembly of split-reporter molecule by protein splicing or complementation, which was previously developed by the present inventors, (Pamphlet of International Publication WO2002/08766 and WO2004/104222, Kim, S. B.; Ozawa, T.; Watanabe, S.; Umezawa, Y. (2004). Proc. Natl. Acad. Sci. U.S.A. 101, 11542-11547. and Kim, S. B., Awais, M., Sato, M., Umezawa, Y., and Tao, H. (2007). Anal. Chem. 79, 1874-1880.) enables determination by simple comparison between inactive split-reporter molecule (without stimulator) and reconstituted active reporter molecule (with stimulator) which dispense with measurement of subtle difference in energy transfer. However, in this method, since each split-reporter molecule is introduced into a cell sign as an individual probe (two-molecule-format probe), expression amount disadvantageously differs between these probes. Also, two probes attempted to be cotransfected to a single cell actually resulted in occurrence of considerable number of cells transfected with either one of them, thus inefficacy of probe set has been strongly concerned. Further, to reconstruct a split-reporter molecules, which are individually introduced into a cell as two probes, into one active molecule, both components consisting of the probe set should sufficiently be approximated; however, to achieve this, it is required for a sufficient affinity between two proteins linked to each fragment of split-reporter molecule, and relatively weak affinity between the proteins hampers efficient analysis of a ligand, which was also problem.

The preceding invention made by the present inventors (Japanese Patent Application No. 2007-005144) was devised to solve the problems as described above, and a novel bioanalytical method providing a single-molecule-format bioluminescent probe in the form of one molecule wherein two proteins having binding ability are sandwiched between split-reporter fragments. However, this approach is insufficient for analysis of in vivo signal in complex context of living cells because it can address only one molecular signal, and only provides information about one-dimensional luminescence intensity. That is, despite inside of a living cell is a complex system, it has the following problems: (i) it fails to provide follow-up means for multiple signals, and (ii) it fails to analyze two-dimensional information such as property and bioactivity level of a ligand. For example, when a cell is stimulated by a ligand such as an anticancer substance or new drug candidate, the cell simultaneously triggers multiple signals in response to the stimulation, and a response protein thereof immediately chooses an optimum signal transduction from the several options. Such endogenous signaling pathways can hardly be traced only by a measure reporting one-dimensional luminescence intensity as is disclosed in the preceding invention; therefore, development of a luminescent probe capable of following up multiple signals and properties at once is strongly demanded.

### DISCLOSURE OF THE INVENTION

### Problem to be Solved by the Invention

The present invention provides a novel ligand detection means capable of exhibiting two-dimensional information (wavelength and intensity of luminescence signal) responding to multiple signals triggered by protein-protein interactions activated by a ligand, while taking advantage of the merit of the aforementioned single-molecule-format bioluminescent probe.

### Means for Solving the Problems

To solve aforementioned problems, the present inventors focused on the fact that photoproteins emit colors with different wavelengths, and conceived of split photoproteins which emit lights with different wavelengths, inserting these into conventional single-molecule-format bioluminescent probes respectively, and using the probes in a set. The split photoprotein temporarily loses its activity due to being dissected. The present inventors found that it enabled the split photoproteins to emit multiple colors according to their multiple recombinations, which were initially provoked by conformational change of an intended ligand recognition protein, and consequently property and bioactivity level of a test substance as a ligand can be analyzed two-dimensionally (i.e., luminescence intensity and wavelength). In an embodiment of the present invention, click beetle luciferases (CBLucs) emitting red and green light were utilized, which are typical pH-insensitive photoproteins. Using a set of two kinds of single-molecule-format bioluminescent probes respectively comprising N- and C-terminal fragments of green luminescent CBLucs and red luminescent CBLucs on their both ends, and by varying a molecular recognition domain region to which a ligand recognition protein in each probe binds depending on conformational change, difference in conformational change of protein can be converted into intensities of green and red luminescence. Technical hurdle in constructing this set of single-molecule-format bioluminescent probes are as follows: (i) a single-molecule-format probe emitting red luminescence as a component of the multicolor probe set cannot be constructed because there is no prior art about the technique of luminescence-based detection of protein phosphorylation occurring in a single molecule; and (ii) when a single cell is cotransfected with two types of single-molecule-format bioluminescent probes, there is a fear that cross interaction may occur between these two molecules. As to a solution for the technical hurdles (i), the inventors made trial-and-error and finally found that by extracting only a phosphorylation recognition site of a kinase (SH2 domain) and connecting it to the ligand recognition protein; thereby phosphorylation of a ligand recognition protein of interest can be reversibly detected in a single molecule. As to a solution for the technical hurdle (ii), through a series of control tests, the inventors found that intramolecular protein-protein binding in a single molecule is of the most prior among any other binding choices including intermolecular binding cases even when various kinds of probe molecules coexist, and accomplished the present invention.

Further, the inventors attempted to create a single-molecule-format multicolor bioluminescent probe in which respective fragment of split green- and red luminescent CBLucs, and every component of two distinctive kinds of molecular recognition domains respectively binding the protein of interest having variable structures depending on the ligand property are tandemly arranged in a single molecular backbone while the ligand recognition protein being centered therein. Since the ligand recognition protein of interest binds a particular one of the two molecular recognition domains situated in different positions in a single molecular backbone in response to the property and bioactivity of a ligand, tandemly-fused luciferase fragments in the same molecule are accordingly approximated, and respective colors of luminescence by the distinctive combination can be observed.

To create such a probe wherein different components are tandemly arranged, it is necessary to arrange a number of components in the probe in an optimum order, and to determine optimal dissection points of green and red luminescent CBLucs which can be applicable in a single-molecule-format. Additionally, to exhibit different colors of red and green luminescence through a variety of protein-protein combination patterns in a single molecular backbone, four split fragments (N-LE-1, C-LE-1, N-LE-2, C-LE-2) are required. However, it is impossible to realize all of these four split fragments with keeping optimal configuration of multicolor probe by employing any arrangement in a single-molecular backbone where a ligand recognition protein located at the center. In other words, it is impossible to make arrangement so that N-LE-1 and C-LE-1 necessarily bind to each other (red luminescence) or N-LE-2 and C-LE-2 necessarily bind to each other (green luminescence) while allowing optimal binding between one of the two molecular recognition proteins and a conformation-changed ligand recognition protein in the center of the molecular backbone. Therefore, it is necessary to remove one luciferase fragment of the four split fragments obtained from the above photoprotein.

The present inventors made earnest study to find that the C-terminal fragments of green luminescent CBLuc has the same property functionally as that of the C-terminal fragment of red luminescent CBLuc among the split four fragments. In light of this, the inventors designed a C-terminal fragment to bind to either of N-terminal fragments of green luminescence or red luminescence, so that the differences of the combinations introduce emission of different colors of luminescence. As described above, the present invention can provide a single-molecule-format multicolor probe, wherein a single molecule backbone allows an intramolecular protein-protein combination, and accordingly combination of split luminescent LEs located adjacent to the proteins varies, and the present invention was accomplished.

As described above, use of a multicolor bioluminescent probe set and a single-molecule-format multicolor probe provided by the present invention enables discretion and detection of property and bioactivity of target ligand in a complex system of a living cell two-dimensionally (wavelength vs. intensity) with multicolor, and two distinctive activities (such as anticancer/carcinogenic activity, agonist/antagonist action and the like) of ligand represented by biologically active agent can be quantitatively assessed in a short time as two-dimensional information with different colors respectively.

The present invention also provides an expression vector capable of expressing the aforementioned probe in a living cell.

The present invention also provides a kit for analyzing ligand activity qualitatively and quantitatively, comprising multicolor bioluminescent probe set or a living cell expressing single-molecule-format multicolor probe; further, the present invention provides a kit for the same by combining the multi color bioluminescent probe set or single-molecule-format multi color probe itself, or an expression vector comprising genes encoding the bioluminescent proves with LE substrate.

The present invention also provides a method for screening an unknown antagonist/agonist that binds the ligand of interest recognition protein and a kit therefor.

More specifically, the present invention is as follows.

[1] A multicolor luminescent probe set that emits two-dimensional luminescence signals of wavelength and intensity in response to a property and level of bioactivity of a ligand, comprising:

(1) a single-molecule-format bioluminescent probe that emits the light of the first wavelength, wherein a fusion protein comprising the first molecular recognition domain and a ligand recognition protein which becomes capable of binding said first molecular recognition domain due to the first conformational change sandwiched between the split N-terminal fragment (N-LE-1) and C-terminal fragment (C-LE-1) of the first Lighting Enzyme (LE-1); and

(2) a singl-moleucle-format bioluminescent probe that emits the light of the second wavelength, wherein a fusion protein comprising the second molecular recognition domain and said ligand recognition protein which becomes capable of binding said second molecular recognition domain due to the second conformational change sandwiched between the split N-terminal fragment (N-LE-2) and C-terminal fragment (C-LE-2) of the second Lighting Enzyme (LE-2).

[2] The multicolor luminescent probe set according to the foregoing [1], wherein the first Lighting Enzyme is a luciferase emitting green luminescence (Luc-Green) and the wavelength of the first luminescence signal is within green range, and the second Lighting Enzyme is a luciferase emitting red luminescence (Luc-Red) and the wavelength of the second luminescent signal is within red range.
[3] The multicolor luminescent probe set according to the foregoing [1] or [2], further comprising a single-molecule-format bioluminescent probe that emits light of the third wavelength, wherein a fusion protein comprising the third molecular recognition domain and the ligand recognition protein either the same or different from said ligand recognition protein which becomes capable of binding said third molecular recognition domain due to the third conformational change sandwiched between the split N-terminal fragment (N-LE-3) and C-terminal fragment (C-LE-3) of the third Lighting Enzyme (LE-3).
[4] The multi color luminescent probe set according to the foregoing [3], wherein the third Lighting Enzyme is a luciferase emitting yellow luminescence (Luc-Yellow) and the wavelength of the third luminescence signal is within yellow range.
[5] The multicolor luminescent probe set according to any one of the foregoing [1]-[4], wherein the ligand recognition protein is the nuclear receptors (NRs), cytokine receptors, or various protein kinases that recognizes a hormone, chemical substance or protein for signal transduction as a ligand.
[6] The multicolor luminescent probe set according to the foregoing [5], wherein the ligand recognition protein is the nuclear receptors (NRs) selected from the group consisting of estrogen receptors (ERs), glucocorticoid receptors (GRs), androgen receptors (ARs), and progesterone receptors (PRs).
[7] The multicolor luminescent probe set according to the foregoing [5] or [6], wherein the first molecular recognition domain is derived from a coactivator, and the second molecular recognition domain is the protein phosphorylation recognition domain when the ligand recognition protein is the nuclear receptors (NRs).
[8] The multicolor luminescent probe set according to the foregoing [7], wherein the molecular recognition domain derived from a coactivator is a molecular recognition domain comprising LXXLL motif, and the protein phosphorylation recognition domain is SH2 domain derived from kinase.
[9] The multicolor luminescent probe set according to any one of the foregoing [1]-[8], further comprising a single-molecule-format bioluminescent probe that emits light of the n-th wavelength, wherein a fusion protein comprising n-th molecular recognition domain and the ligand recognition protein either the same or different from said ligand recognition protein which becomes capable of binding to said n-th molecular recognition domain due to the conformational change sandwiched between the split N-terminal fragment (N-LE-n) and C-terminal fragment (C-LE-n) of the n-th Lighting Enzyme (LE-n), provided that n is a natural number of 3 or larger.
[10] A single-molecule-format multicolor bioluminescent probe that emits two-dimensional luminescence signals of wavelength and intensity in response to property and level of bioactivity of a ligand, wherein a fusion protein comprising the first molecular recognition domain and a ligand recognition protein which becomes capable of binding said first molecular recognition domain due to the first conformational change sandwiched between the split N-terminal fragment (N-LE-1) and C-terminal fragment (C-LE-1) of the first Lighting Enzyme (LE-1); wherein the second molecular recognition domain bound by said ligand recognition protein which acquired a binding capability due to the second conformational change is connected further to the N-terminal side of the N-LE-1, and further to the N-terminal side thereof the split N-terminal fragment (N-LE-2) of the second Lighting Enzyme (LE-2) is connected; wherein self-complementation between N-LE-1 and C-LE-1 takes place when the first molecular recognition domain is bound by the ligand recognition protein due to the first conformational change as a result of binding of the ligand by the ligand recognition protein, and triggers emission of the luminescence signal of the first wavelength; in like wise, the second molecular recognition domain is bound by the ligand recognition protein due to the second conformational change, followed by the self-complementation between N-LE-2 and C-LE-1 takes place, and thereby triggers emission of the luminescence signal of the second wavelength.
[11] The single-molecule-format bioluminescent probe according to the foregoing [10], wherein the first Lighting Enzyme is luciferase emitting green light (Luc-Green) and wavelength of the first luminescence signal is within green range, and the second Lighting Enzyme is luciferase emitting red light (Luc-Red) and wavelength of the second luminescence signal is within red range.
[12] The single-molecule-format bioluminescent probe according to the foregoing [10] or [11], wherein the ligand recognition protein is the nuclear receptors (NRs), cytokine receptors, or various protein kinases.
[13] The single-molecule-format multicolor luminescent probe according to the foregoing [12], wherein the ligand recognition protein is the nuclear receptors (NRs) selected from the group consisting of estrogen receptors (ERs), glucocorticoid receptors (GRs), androgen receptors (ARs), and progesterone receptors (PRs).
[14] The single-molecule-format multicolor bioluminescent probe according to any one of the foregoing [10]-[13], wherein the first molecular recognition domain is derived from a coactivator, and the second molecular recognition domain is the protein phosphorylation recognition domain.
[15] The single-molecule-format multicolor bioluminescent probe according to the foregoing [14], wherein the molecular recognition domain derived from a coactivator is a molecular recognition domain comprising LXXLL motif, and the phosphorylation recognition domain is SH2 domain derived from a kinase.
[16] A set of nucleic acids encoding multicolor bioluminescent probe set that emits two-dimensional luminescent signals of wavelength and intensity in response to property and bioactivity level of a ligand; and capable of expressing the multicolor luminescent probe set of any of the foregoing [1]-[9] in a living cell line comprising:

(1) the nucleic acids encoding a single-molecule-format bioluminescent probe that emits light of the first wavelength, wherein the nucleic acids encoding a fusion protein comprising the first molecular recognition domain and a ligand recognition protein which becomes capable of binding said first molecular recognition domain due to the first conformational change sandwiched between the nucleic acids encoding the split N-terminal fragment (N-LE-1) and C-terminal fragment (C-LE-1) of the first Lighting Enzyme (LE-1); and

(2) the nucleic acids encoding a singl-moleucle-format bioluminescent probe that emits the light of the second wavelength, wherein the nucleic acids encoding a fusion protein comprising the second molecular recognition domain and said ligand recognition protein which becomes capable of binding said second molecular recognition domain due to the second conformational change sandwiched between the nucleic acids encoding the split N-terminal fragment (N-LE-2) and C-terminal fragment (C-LE-2) of the second Lighting Enzyme (LE-2).

[17] An expression vector wherein a set of nucleic acids encoding the multicolor luminescent probe set according to the foregoing [16] is inserted or a set of expression vectors wherein each of the nucleic acids are inserted, and said single expression vector or said expression vector set which is capable of expressing a multicolor luminescent probe set that emits two-dimensional luminescent signals of wavelength and intensity in response to property and bioactivity level of a ligand.
[18] A transfected living cell line, wherein the expression vector or the vector set according to the foregoing [17] is inserted, thereby expressing a multicolor luminescent probe that emits two-dimensional luminescent signals of wavelength and intensity in response to property and bioactivity level of a ligand.
[19] Nucleic acids encoding a single-molecule-format multicolor bioluminescent probe that emits two-dimensional luminescent signal of wavelength and intensity in response to property or bioactivity level of a ligand, wherein the nucleic acids encoding the single-molecule-format multicolor bioluminescent probe according to the foregoing [10]-[15], wherein the nucleic acids encoding a fusion protein of the first molecular recognition domain and the ligand recognition protein sandwiched between nucleic acids encoding split N- and C-terminal fragment of the first Lighting Enzyme (LE-1), wherein the nucleic acids encoding the second molecular recognition domain are connected further to the N-terminal side of the nucleic acids encoding N-LE-1, and further to the N-terminal side thereof are connected the nucleic acids encoding the split N-terminal fragment (N-LE-2) of the second Lighting Enzyme (LE-2), wherein self-complementation between N-LE-1 and C-LE-1 takes place when the first molecular recognition domain is bound by the ligand recognition protein as a result of the first conformational change, triggers the emission of the luminescent signal of the first wavelength; whereas self-complementation between N-LE-2 and C-LE-1 takes place when the second molecular recognition domain is bound by the ligand recognition protein as a result of the second conformational change, triggers the emission of the luminescent signal of the second wavelength.
[20] An expression vector comprising the nucleic acids encoding a single-molecule-format multicolor bioluminescent probe according to the foregoing [19], wherein the expression vector is capable of expressing a single-molecule-format multicolor bioluminescent probe that emits two-dimensional luminescent signals of wavelength and intensity in response to property and bioactivity level of a ligand in a living cell line.
[21] A living cell line expressing a single-molecule-format multicolor bioluminescent probe that emits two-dimensional luminescent signal with wavelength and intensity in response to property and bioactivity level of a ligand, wherein the living cell line is transfected with the expression vector according to the foregoing [20], comprising the nucleic acids encoding a single-molecule-format multicolor bioluminescent probe.
[22] A method for qualitative and quantitative evaluation of the activity of test substance as a ligand for the subject protein, wherein the method comprises the step of stimulating a multicolor luminescent probe set expressed in a living cell line according to the foregoing [18] or single-molecule-format multicolor bioluminescent probe expressed in a living cell according to the foregoing [21] respectively with the test substance; followed by measuring the wavelength and intensity of luminescence; and thereby analyzing the test substance for property and level of bioactivity.
[23] A method for determining antagonist/agonist activity of a test substance, wherein the method comprises the steps of stimulating a multicolor luminescent probe set expressed in a living cell according to the foregoing [18] or single-molecule-format multicolor bioluminescent probe expressed in a living cell according to the foregoing [21] with the test substance; followed by evaluating each lighting enzyme with the changes in luminescence intensity ratios before and after the stimulation.
[24] A method of screening antagonist and/or agonist against the subject ligand recognition protein, wherein the method comprises the steps of stimulating the multicolor luminescent probe set expressed in a living cell according to the foregoing [18] or the single-molecule-format multicolor bioluminescent probe expressed in a living cell according to the foregoing [21] with the test substance; followed by measuring wavelength and intensity of the luminescence.
[25] A kit for qualitative and quantitative evaluation of the activity of the test substance as a ligand for the subject protein, comprising the living cell line which expresses the multicolor luminescent probe set according to the foregoing [18] or the single-molecule-format multicolor bioluminescent probe according to the foregoing [21].
[26] A kit for screening antagonist and/or agonist against the ligand recognition protein, comprising the living cell lines which expresses the multicolor luminescent probe set according to the foregoing [18] or the living cell line which expresses the single-molecule-format multicolor bioluminescent probe according to the foregoing [21].
[27] A kit for qualitative and quantitative analysis for the ligand activity or a kit for screening antagonist and/or agonist against the ligand recognition protein, wherein combined a Lighting Enzyme substrate used for each luminescent probe with the single expression vector or the set of expression vectors according to the foregoing [17] which can express the multicolor luminescent probe set in a living cell, or the expression vector according to the foregoing [20] which comprises the nucleic acids encoding a single-molecule-format multicolor luminescent probe.
[28] A kit for qualitative and quantitative analysis for ligand activity or a kit for screening antagonist and/or agonist against a ligand recognition protein, wherein combined a Lighting Enzyme substrate used for each luminescent probe with the multicolor luminescent probe set according to any of the foregoing [1]-[9] or the single-molecule-format multicolor luminescent probe according to any one of the foregoing [10]-[15].

### EFFECTS OF THE INVENTION

The present invention provides novel means for detecting the presence/absence of multiple bioactivities (pharmacological activity, toxicity, carcinogenicity etc.) of a target-specific ligand including, for example, drugs, toxins, and carcinogens as a multicolor and two-dimensional spectrum simply, rapidly and accurately. The present invention also provides a basic technique for screening unknown antagonist/agonist for a wide range of “ligand recognition proteins” by using living cell lines transformed to express a multicolor bioluminescent probe set or a single-molecule-format multicolor bioluminescent probe via an expression vector therefore.

Consequently, multiple signal transductions in cell lines and living subjects are simultaneously visualized, and precise bioanalysis using wavelengths and luminescence intensity as indexes can be performed. The present invention further provides a probe which enables evaluation of multiple bioactivities of a ligand, which was not realized by any conventional method, and also capable of providing two-dimensional information of multiple signals rapidly and simply at high sample throughput. The present invention is useful for high speed screening for risk factors in a living subject such as carcinogens and rapid quantitative evaluation of pharmaceutical action of anticancer agents (development of new drugs).

### BRIEF DESCRIPTION OF THE DRAWINGS

FIG. 1 is a schematic illustration showing the sensing mechanism of a single-molecule-format probe set and a single-molecule-format multicolor bioluminescent probe. The number 1 shows a red luminescent probe for detecting anticancer ability of a ligand. The probe is activated based on ligand-dependent binding between ER LBD and SH2 domain of carcinogenesis regulation protein Src which is a protein phosphorylation recognition domain for ER LBD. The subsequent red luminescence serves as an index of nongenomic activity of the ligand. On the other hand, the number 2 shows a probe which is activated by binding between ER LBD stimulated by a ligand and LXXLL motif of Src-1a of a coactivator. Accordingly, this probe emits green luminescence which is index of genomic activity of the ligand. The number 7 shows a probe in which the green- and red-luminescence-emitting probes are integrated in a single-molecule-format, wherein the combination between LE fragments varies depending on the property of a ligand of interest, thereby emitting multicolor luminescence. The characters “N” and “C” used herein respectively indicate an N— and a C-terminal side fragments of LE.

FIG. 2 is a schematic diagram of respective luminescent probe constructs. The diagram shows backbones of respective cDNA constructs of pSimer-R series probe, pSimer-G series probe, and pSimer-RG series probe.

FIG. 3 shows determination of optimal dissection points of CBLuc-Red which is applicable to red luminescent probe (pSimer-R series) for phosphorylation measurement.

FIG. 4 shows response spectrum of a cell carrying pSimer-R2 in response to various ligands with all wavelengths.

FIG. 5 shows ligand response (luminescence intensity) of each cell line transfected with pSimer-R2.

FIG. 6 shows change in ligand-dependent red-luminescence intensity with time in COS-7 cells transfected with pSimer-R2.

FIG. 7 shows change in ligand sensitivity of COS-7 cells transfected with pSimer-R2m wherein phosphorylation site (537 AA) of estrogen LBD (ER LBD) is deficient.

FIG. 8 shows determination of optimum LXXLL motif capable of binding ER LBD.

FIG. 9 shows a ligand-dependent luminescence intensity spectrum of COS-7 cells transfected with pSimer-G6.

FIG. 10 shows a determination of dose-response curves of pSimer-G4 and pSimer-G6 which are convincing plasmids of pSimer-G series.

FIG. 11 shows ligand-response spectrum of COS-7 cells cotransfected with pSimer-R2 plasmid and pSimer-G4 plasmid with all wavelengths.

FIG. 12 shows ligand-response spectrum of COS-7 cells cotransfected with pSimer-R2 plasmid and pSimer-G6 plasmid with all wavelengths.

FIG. 13 shows ligand-dependent spectrum of COS-7 cells transfected with a pSimer-RG series plasmid with all wavelengths. a and b show spectrum of COS-7 cells transfected with pSimer-RG1 and pSimer-RG2, respectively.

FIG. 14 shows schematic diagram of ligand response, in a cell, of various single-molecule-format multicolor bioluminescent probes described in the present invention. a is a schematic diagram of ligand response in the cytosol by parallel conformational changes of the probes when pSimer-R series probe and pSimer-G series probe (a single-molecule-format bioluminescent probe set) are cotransfected, and b shows response by parallel conformational changes of the probe in the cytosol when pSimer-RG series probe (a single-molecule-format multicolor bioluminescent probe) is transfected.

### THE BEST MODE FOR CARRYING OUT THE INVENTION

The present invention relates to a set of multiple kinds of conventional luminescent probes with different wavelengths or a single-molecule-format multicolor bioluminescent probe.

A multicolor bioluminescent probe set of the present invention, each bioluminescent probe is comprised of fusion protein, wherein (i) a ligand response domain and (ii) a molecular recognition domain, which binds reversibly to the ligand response domain, sandwiched between dissected Lighting Enzymes (LE), and each bioluminescent probe emits light with different wavelengths in response to conformational changes of various ligand recognition proteins in a living cell.

The “single-molecule-format multicolor probe” herein refers to a probe optimally integrated with components capable of visualizing multiple activities of a target-specific ligand as multiple bioluminescent in a single fusion molecule. Particularly, a fusion protein comprised of (i) ligand-response domain, and (ii) molecular recognition domain which binds reversibly to the ligand-response domain (for example, SH2 domain of Src) as well as (iii) three dissected fragment LE (two distinct N-terminal side fragments and one C-terminal side fragment in common), which is expressed in a cell by an expression vector to which chimera DNA with tandemly-arranged DNA encoding each proteins is inserted. “Chimera DNA” used herein means a DNA comprised of tandemly connected multiple short pieces of DNA derived from different origins, which can express a fusion protein molecule comprised of proteins (peptides) as expression products of the DNA.

The term “split/dissected Lighting Enzyme (LE)” used herein means that a single molecule LE is dissected into two molecules and thereby temporarily being inactivated (the activity is temporarily lost). The dissection point is hydrophilic site, wherein LE is dissected at appropriate point for reconstitution so that the two split LEs can restore the activity (restore the luminescence activity) by self-complementation when they are approximated.

These components are integrated into an expression vector as chimera DNA in which respective DNA pieces encoding each component are directly and tandemly arranged or DNA encoding optimal “linker peptide” is mediated therein.

A living cell line is transformed with an expression vector containing a set of chimera DNA encoding the multicolor bioluminescent probe set or chimera DNA encoding the single-molecule-format multicolor bioluminescent probe of the present invention, and property and bioactivity level of a test substance as a ligand for the ligand recognition protein in the probe are analyzed by using the multicolor bioluminescent probe set or the single-molecule-format multicolor bioluminescent probe expressed in the living cells.

When the multicolor bioluminescent probe set is expressed in a living cell, nucleic acids encoding the respective bioluminescent probes may be introduced into the living cell via a single expression vector or vectors comprising respective nucleic acids may be simultaneously introduced into the same living cell.

In the present invention, the term “living cells” means culture cells maintaining its natural function or eukaryotic cells present in living individuals (yeast cells, insect cells, animal cells), particularly mammalian cells including human cells. The term “living cells” also includes prokaryotic cells.

As the “expression vector” of the present invention, any known expression vectors for eukaryotic or prokaryotic cells can be used without particular limitation. Further, in order to control expression of the chimera DNA (for example, in specific tissue in a living individual), a known plasmid containing tissue-specific promoter sequence may be used. A probe expression vector may be introduced into a cell by known transfection techniques such as micro-injection method, electroporation method and the like. Alternatively, intracellular introduction method using a lipid (BioPORTER (Gene Therapy Systems, US), Chariot (Active Motif, US) etc.) may be employed.

In the following, each component of bioluminescent probe is explained.

The term “ligand recognition protein” used herein refers to a protein that responds to an extracellular stimulation and undergoes a conformational change in response to ligand stimulation, thereby binding molecular recognition domain of other proteins or coactivators. Examples of such “ligand recognition protein” used herein include nuclear receptors (NRs), cytokine receptors or various protein kinases, for which hormones, chemical substances or signaling proteins (e.g., cytokines, chemokines, insulin) recognized as ligands. In particular, nuclear receptor ligand binding domains (NR LBDs) are preferable, and estrogen receptors (ERs) recognizing estrogens as ligands, glucocorticoid receptors (GRs), androgen receptors (ARs) or progesterone receptors (PRs) are preferably used. When intracellular second messengers or lipid second messengers are used as ligands, binding domains of respective second messengers may be employed.

Estrogen receptor LBDs described in an embodiment may be used by preparing the LBD regions thereof (305-550 AA) through genetic engineering or PCR method based on sequence information of entire sequence of human ERs (GenBank/P03372). LBDs of nuclear receptors, for example LBDs of androgen receptors (AR LBDs) can be used by preparing the LBD regions thereof (672-910 AA) through genetic engineering or PCR method based on sequence information of entire sequence of human ARs (GenBank/AF162704). LBDs of human glucocorticoid receptors (GR LBDs) may also be used by preparing the LBD regions thereof (527-777 AA) based on sequence information of entire sequence of human GRs (GenBank/1201277A). Further, human mineral corticoid receptors (MR; GenBank/P08235) and human progesterone receptors (PR; GenBank/P06401) may be used as ligand recognition proteins of the multicolor bioluminescent probes of the present invention.

The term “ligand” used herein means a substance capable of specifically binding a ligand recognition protein molecule in a living cell line and thereby changing function thereof. It is, for example, agonists or antagonists for receptor proteins (for example, nuclear receptors or G-protein coupled receptors). In another embodiment, signal transduction proteins such as cytokines, chemokines and insulin, which specifically bind molecules involved in signal transductions in a cell line, intracellular second messengers, lipid second messengers, phosphorylated amino acid residues, or ligands for G protein coupled receptors are preferred.

The term “phosphorylation recognition domains” used herein refers to domains which can recognize phosphorylated amino acid residues, and the domains correspond to sites called “SH2 domains” in various kinase proteins. In an embodiment of the present invention, phosphorylation recognition domains domain; 150-248 AA) of proto-oncogene tyrosine-protein kinase, Src, (GenBank/NP938033) is used; however, other SH2 domains may also be used. For example, SH2 domains of growth factor receptor-binding protein 2, Grb2, involved in cell proliferation, carcinogenesis and the like are also preferably used.

Further, as peptide sequences capable of binding ER LBDs of the present invention, LXXLL motifs of coactivators are typically used. Preferably, LXXLL motifs (about 15 AA) of Rip140 (GenBank/NP003480), which are one kind of coactivators, or Src-1a (steroid receptor coactivator 1 isoform 1; GenBank/NP003734) is used. As other peptide sequences capable of binding ER LBDs, FXXLF motifs, WXXLF motifs and the like may be used.

Among the photoproteins of the present invention, Lighting Enzymes (LE), “which can be dissected in N— and C-terminal side fragments (respectively called N-LE and C-LE),” and firefly luciferases (FLucs), Renilla luciferases (RLucs), Gaussia luciferases (GLucs), Click Beetle luciferases (CBLucs) and the like are preferred. CBLucs emitting two colors of light, green light and red light, were used in the present invention. Biouminescent probes emitting three colors of light can also be constructed by incorporating firefly luciferases (FLuc) emitting yellow luminescence.

Amino acids and the cDNA sequences of the LEs are known, for example, FLuc (GenBank/AB062786 etc.), CBLuc (GenBank/AY258592.1 etc.) and the like; therefore, DNA of the LEs can be obtained by know methods based on such sequence information. The dissection points of these LEs can be determined accordingly by reference to such as known information. For example, FLuc can be dissected at 437/438 AA as described in Paulmurugan, R., and Gambhir, S. S. (2005). Anal. Chem. 77, 1295-1302., while CBLuc can be dissected at 412/413 as shown in examples as follow. As disclosed in Pamphlet of International Publication WO2004/104222, RLucs can be dissected at appropriate points; however, the luminescence intensity after reconstitution is maximum if they are dissected at 91/92 AA. Further, CBLucs can be dissected at 439/440 AA or 412/413 AA. As shown in the following examples, N- and C-terminal side fragments partially overlapped or deficient can be used.

Aforementioned components may be connected in any order provided that the dissected LEs are located on both ends and in between. However, when ER LBD, LXXLL motif, and Src SH2 are used for detecting a ligand of ER, it is necessary to connect N-LE and Src SH2, also C-LE and LBD, as shown in the following examples. Concrete probe configuration is [N-LE/Src SH2/N′-LE/LXXLL motif/ER LBD/C-LE] from the N-terminal side. For the purpose of screening for carcinogens, from this probe only a probe for protein phosphorylation detection may be constructed based on Src which is a carcinogenesis regulation protein. In this case, the probe configuration is [N-LE/Src SH2/ER LBD/C-LE] from the N-terminal side.

If binding between other ligand and a ligand recognition protein is of interest, appropriate connecting order may be employed for each case. Such appropriate orders may be examined, for example, by varying the introduction order of each DNA into a probe expression vector. DNA introduction into an expression vector may be readily performed by those skilled in the art; however, certain degree of trial-and-error is required to determine an appropriate connecting order.

The present probe may have respective components connected by linker peptides. In particular, a LBD and a LBD interacting peptide are preferably connected by a linker peptide (for example, GS linker composed of repeated sequence of glycine and serine) which causes little steric hindrance and having high flexibility (glycine (G), alanine (A), serine (S) etc.), so that the ligand and the LBD are approximated to bind. For the present probe, it is most preferable to connect the components by 5 to 10 GS linkers.

The most preferable order of the components in the single-molecule-format multicolor bioluminescent probe of the present invention is [N-LE/SH2 domain of kinase/N′-LE/LXXLL motif/NR LBD/C-LE] from the N side. N-LE and N′-LE are N-terminal side fragments of Lighting Enzymes respectively emitting different colors, and C-LE is the C-terminal side fragment of the Lighting Enzymes which is common in these enzymes of different colors. These dissected three fragments are allocated at both ends of the probe and in between, and an “LXXLL motif,” which is the first molecular recognition domain of NR LBD upon the first conformational change, and a SH2 domain of kinase, which is the second molecular recognition domain of NR LBD upon the second conformational change, are respectively located at both adjacent sides of N′-LE within the probe. In an embodiment of the present invention, N-terminal side fragment of red-CBLuc was used as N-LE, N-terminal side fragment of green-CBLuc was used as N′-LE, and C-terminal side fragment of red-CBLuc was used as C-LE.

The term “qualitative and quantitative analysis of activity as a ligand” used herein means determining property and bioactivity level as a ligand, wherein a multicolor bioluminescent probe set or a single-molecule-format multicolor bioluminescent probe expressed in a living cell line is stimulated by the test substance (it can be a ligand itself or sample containing the ligand), and how the ligand recognition protein in a bioluminescent probe underwent conformational change, to which the protein connected, that is what type of conformational changes are triggered by the test substance were two-dimensionally assessed and evaluated with wavelength and intensity of light emitted as indexes. By applying this analytical method based on the multicolor bioluminescent probe set or the single-molecule-format multicolor bioluminescent probe to a test substance whose property as a ligand is unknown, it enables screening of agonist/antagonist.

For example, agonistic/antagonistic activity of a test substance for estrogen receptor (ER) is evaluated by intensity of red light and green light which respectively corresponds to binding levels of the phosphorylation recognition domain of ER and the LXXLL motif. Objective quantification is enabled by using the following estrogenicity score calculating method.

Estrogenicity score=change of luminescence intensity at 540 nm/change of luminescence intensity at 610 nm

In each screening method of the present invention, a transformed living cell line with an expression vector comprising nucleic acids encoding multicolor bioluminescent probe set or a single-molecule-format multicolor bioluminescent probe is usually stimulated by the test substance and wavelength and intensity of the emitted light is measured; however, in the absence of a living cell line, in vitro observation also becomes possible by using a culture solution containing a multicolor bioluminescent probe set or a single-molecule-format multicolor bioluminescent probe secreted in the culture medium or using purified multicolor bioluminescent probe set, provided substrates of each Lighting Enzyme are present simultaneously.

The multicolor bioluminescent probe set or the single-molecule-format multicolor bioluminescent probe can be expressed abundantly in prokaryotic cells such as bacteria as well as in eukaryotic cells like mammalian cells as described above. For example, in mammalian cells, culture supernatant containing a large quantity of probes which can be used for the analysis can be obtained without purification steps by connecting an appropriate signal sequence (such as MGVKVLFALICIAVAEA) to the probe and making the probe to be secreted in the medium culture in a large quantity. Further, by attaching a tag for purification (for example, H is Tag; HHHHHH), a large quantity of purified multicolor bioluminescent probe set or single-molecule-format multicolor bioluminescent probe can be obtained. A kit comprising these purified multicolor bioluminescent probe set or single-molecule-format multicolor bioluminescent probe in combination with Lighting Enzyme can be used as a kit for qualitative and quantitative analysis of ligand bioactivity or a kit for antagonist/agonist screening, similarly a kit comprising a living cell line expressing these bioluminescent probes.

For analysis or screening with purified multicolor bioluminescent probe set or single-molecule-format multicolor bioluminescent probe, the following method can be applied; however, it is not limited thereto.

(i) Collect probes secreted from cells from the culture medium and stock them at 4° C. (probes expressed in prokaryotic cells can be collected in about 0.5 mg/L after 48 hours incubation)
(ii) To a culture (100 μL) containing the probes, add coelenterazine, substrate, (20 μL) to the final concentration of 40 μM.
(iii) Add ligand further, and incubate for 20 minutes.
(iv) Measure luminescence intensity.

Expression vectors comprising nucleic acids encoding a multicolor bioluminescent probe set or single-molecule-format multicolor bioluminescent probe also can be combined with substrates of Lighting Enzyme to make the kit.

For analysis or screening with such a kit, the following method can be applied; however, it is not limited thereto.

(i) Into a cell incubated on 10 cm-culture plate, insert expression vectors using a known Lipofection reagent (for example, TransIT-LT1 (Mirus)).
(ii) After 36 hours, stimulate with ligand and leave for 20 minutes.
(iii) Collect the cells, add known Lysis buffer (300 μL), and incubate for 5 minutes.
(iv) To the lysate (100 μL), add coelenterazine (20 μL) to the final concentration of (40 μM), and immediately measure luminescent intensity.

As described above, as a screening kit of the present invention, a kit comprising expression vectors containing nucleic acids encoding a multicolor bioluminescent probe set or single-molecule-format multicolor bioluminescent probe in combination with substrates of respective Lighting Enzyme in the probe as well as a kit comprising a living cell transfected with expression vector comprising nucleic acids encoding multicolor bioluminescent probe set or a single-molecule-format multicolor bioluminescent probe are used.

Other terms and concepts in the present invention are specifically defined in description of the embodiments and in the examples of the invention. Basically terms are based on the IUPAC-IUB Commission on Biochemical Nomenclature or the meanings of terms conventionally used in the art. Various techniques used for carrying out the present invention can be readily and securely executed by those skilled in the art according to known literatures and the like, except for the techniques whose source is indicated. For example, genetic engineering and molecular biology techniques can be performed by methods described in the documents listed below, referred therein, or methods substantially equivalent to such methods or modified methods thereof: J. Sambrook, E. F. Fritsch & T. Maniatis. (1989). Molecular Cloning: A Laboratory Manual (2nd edition). Cold Spring Harbor, N.Y.: Cold Spring Harbor Laboratory Press; D. M. Glover et al. ed. (1995). DNA Cloning 2nd ed. Vol. 1 to 4, (The Practical Approach Series), IRL Press, Oxford University Press; Ausubel, F. M. et al. (1995). Current Protocols in Molecular Biology. New York: John Wiley & Sons, New York; The Japanese Biochemical Society ed. (1986). Biochemistry Experiment Lecture. Second series, Vol. 1, Genetic study method II. Tokyo: TOKYO KAGAKU DOZIN; The Japanese Biochemical Society ed. (1992). New Biochemistry Experiment Lecture. Vol. 2, Nucleic acid III (recombinant DNA technique). Tokyo: TOKYO KAGAKU DOZIN; R. Wu ed. (1980). Methods in Enzymology. Vol. 68 (Recombinant DNA). New York: Academic Press; R. Wu et al. ed. (1983). Methods in Enzylmology. Vol. 100 (Recombinant DNA, Part B) & 101 (Recombinant DNA, Part C) New York: Academic Press; R. Wu et al. ed. (1987). Methods in Enzymology. Vol. 153 (Recombinant DNA, Part D), 154 (Recombinant DNA, Part E) & 155 (Recombinant DNA, Part F). New York: Academic Press, etc. A variety of proteins and peptides used in the present invention and DNAs encoding the same are available from existing database (e.g., URL: http://www.ncbi.nlm.nih.gov/).

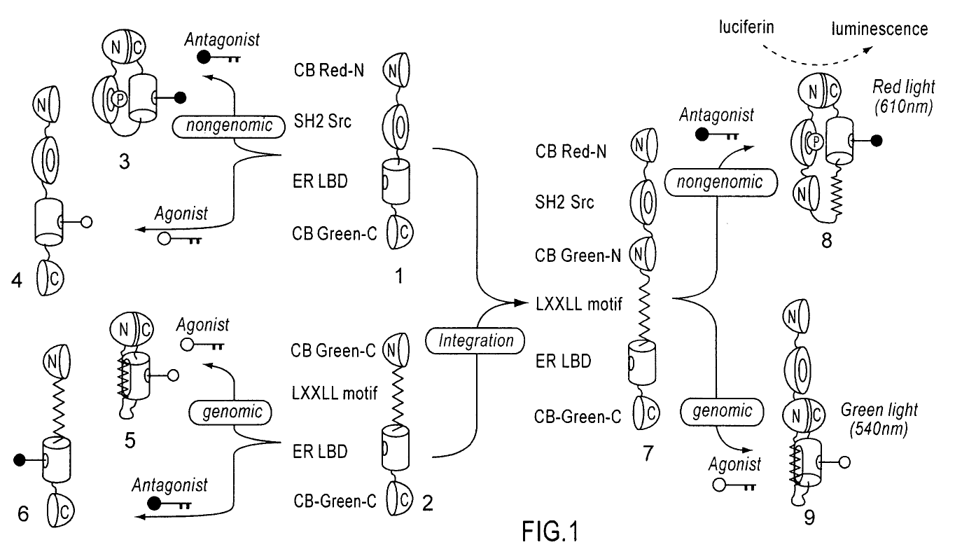

In the following, a detection method of a ligand using pSimer-G series (bioluminescent probe emitting green light), pSimer-R series (bioluminescent probe emitting red light), or pSimer-RG series probe (single-molecule-format multicolor bioluminescent probe which emits red or green light according to the difference in combination of components within a single molecule) is described as an embodiment of the present invention referring to FIG. 1; however, the present invention is not limited thereto. In the example shown in FIG. 1, ER LBD is used as LBD, a LXXLL motif and a SH2 domain of Src are used as the molecular recognition domains, and three fragments obtained by dissecting CBLucs emitting green and red light respectively (two N-terminal side fragments and one C-terminal side fragment) are used as LE. ER LBD and other components are connected via GS linkers.

Without stimulation, any of pSimer-G series, pSimer-R series, and pSimer-RG series are in upright state, interactions between dissected LE fragments do not occur. However, in the presence of antagonistic ligands, only the pSimer-R series probes are activated and emit red light. On the other hand, in the presence of agonistic ligands, only the pSimer-G series probes are activated and emits green light. Some ligands may have activity at both ends, and such ligands emit both of red and green lights. In the probes of pSimer-RG series which is a combination of optimal probes of pSimer-G series and pSimer-R series, the aforementioned phenomena occur in a single molecule.

Ligand-dependent conformational change occurring in probes of each series is reversible, so the linear conformation is restored upon removal of the ligand from ER LBD. Usually, a ligand binds temporarily with its target molecule, so ligands can be detected repeatedly in cells expressing this probe.

Example of test substances for the screening method for these includes organic or inorganic compounds (particularly, low-molecular-weight compounds), proteins and peptides having biological activity and the like. Functions and structures of these substances may be known or unknown. Also, “combinatorial Chemical Library” is a useful means as a group of test substances for effectively identifying the substances of interest. Preparation and screening of combinatorial chemical library are well known in the art (see, for example, U.S. Pat. No. 6,004,617; No. 5,985,365). Further, commercially available libraries (made by, for example, ComGenex, U.S.; Asinex, Russia; Tripos, Inc. U.S.; ChemStar, Ltd, Russia; 3D Pharmaceuticals, U.S.; Martek Biosciences, U.S.) may be used. Also, by applying combinatorial chemical library to a cell population that expresses the present probe, a so-called “high-throughput screening” can be carried out.

### EXAMPLE

In the following, the present invention is explained further in detail and concrete by the following examples; however, the invention is not limited to the examples.

### Example 1

### Construction of Plasmid Encoding Single-Molecule-Format Bioluminescent Probe Set

### (1-1) Construction of Nucleic Acids Encoding Single-Molecule-Format Bioluminescent Probe Emitting Red Light by Phosphorylation-Dependent Protein-Protein Interaction

cDNAs of ER LBD (305-550 AA) and phosphorylation-recognition domain of Src (SH2) were amplified by PCR, and specific restriction sites were introduced at both ends. Recombinant DNAs in which cDNAs encoding ER LBD and Src SH2 were connected to each other were produced by ligation. Subsequently, the cDNAs encodes full CB Red were fragmented at five different points roughly localizing a sequence region encoding hydrophilic amino acids around 4/5 from the beginning of the cDNAs. As a result, five sets of cDNA fragments of CB Red (CB Red-N and CB Red-C) were created. Between the N- and C-terminal fragments (CB Red-N and CB Red-C, respectively) of these five sets, the connected components, Src SH2-ER-LBD, were inserted. Consequently, DNA constructs [CB Red-N/Src SH2/ER LBD/CB Red-C] were produced and sub-cloned into pcDNA3.1(+) vector backbone (Invitrogen). The sequences of these constructed plasmids were verified by using BigDye Terminator Cycle Sequencing kit and ABI Prism310 Genetic Analyzer. This plasmid series was named pSimer-R. According to the different cutting sites, these plasmids were named from pSimer-R1 to pSimer-R5.

### (1-2) Construction of Nucleic Acids Encoding Single-Molecule-Format Bioluminescent Probe Emitting Green Light by Protein-Protein Binding Between Coactivators and Er LBD

From the full-length cDNA encoding CB Green, the N-terminal side cDNA fragment encoding 1-439 amino acids (CB Green-N) and the C-terminal side cDNA fragment encoding 440-542 amino acids (CB Green-C) were created by PCR. As representatives of coactivators, total six kinds of cDNA fragments encoding LXXLL motifs of Rip140 and Src1 were generated (table 1), and cDNA of the above-described ER LBD were respectively connected with the six cDNA fragments. Subsequently, the cDNA constructs encoding the LXXLL motif-ER LBD were introduced in between CB Green-N and CB Green-C to create chimera DNA and sub-cloned into pcDNA3.1(+) vector backbone (Invitrogen). The DNA sequences of these constructed plasmids were verified by using BigDye Terminator Cycle Sequencing kit and ABI Prism310 Genetic Analyzer. This plasmid series was named pSimer-G.

TABLE 1
Amino acid sequence of LXXLL motif derived from
coactivators applied to pSimer-G series probes

| | Source | |
|---|---|---|
| Name | (name of coactivator) | Amino acid sequence |
| pSimer-G1 | Src1 | 272 ARHKILHRLLQEGSP 287 |
| pSimer-G2 | Src1 | 287 PSGEQLLRHLIKHRA 272 |
| pSimer-G3 | Rip140 | 495 HQKVTLLQLLLGHKN 509 |
| pSimer-G4 | Rip140 | 509 NKHGLLLQLLTVKQH 495 |
| pSimer-G5 | Rip140 | 932 SFNVLKQLLLSENCV 946 |
| pSimer-G6 | Rip140 | 946 VCNESLLLQKLVNFS 932 |

### (1-3) Construction of Nucleic Acids Encoding Single-Molecule-Format Multicolor Bioluminescent Probe

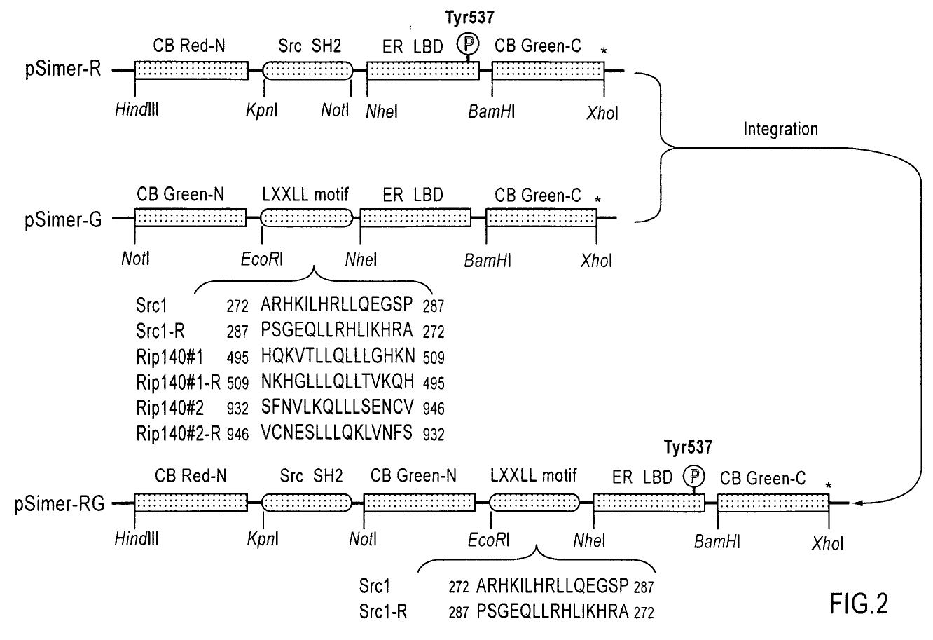

A single-molecule-format multicolor bioluminescent probe was constructed based on the backbones of the above-described pSimer-R series and pSimer-G series. cDNA encoding CB Red-N-Src SH2 were cut out from the above-descrived pSimer-R2, and cDNA encoding CB Green-N-LXXLL motif, ER LBD-CB Green-C were cut out from the pSimer-G6, and subsequently chimera cDNA shown in FIG. 2 was generated.

### Example 2

### Gene Tansfection of Plasmid Expressing Single-Molecule-Format Bioluminescent Probe into Living Cells

COS-7 cells derived from African Green Monkey's kidney were cultured in Dulbecco-modified Eagle's medium (DMEM; Sigma) supplemented with 10% steroid-deficient fetal bovine serum (FBS) and 1% penicillin-streptomycin (P/S), and incubated in a 12-well plate in a cell incubator at 37° C. and 5% CO₂. Using TransIT-LT1 (mirus), commercially available gene transfection kit pSimer-G, pSimer-R, or pSimer-RG series plasmids were transfected into COS-7 cells in the 12-well plate. The COS-7 cells containing respective plasmids were incubated in a cell incubator for 16 hours, allowed to sufficiently express respective bioluminescent probes, and used in the following experiments.

### Example 3

### Determination of Optimal Dissection Site in CBLuc-Red for Red Luminescent Probe Used for Measuring Protein Phosphorylation (pSimer-R Series)

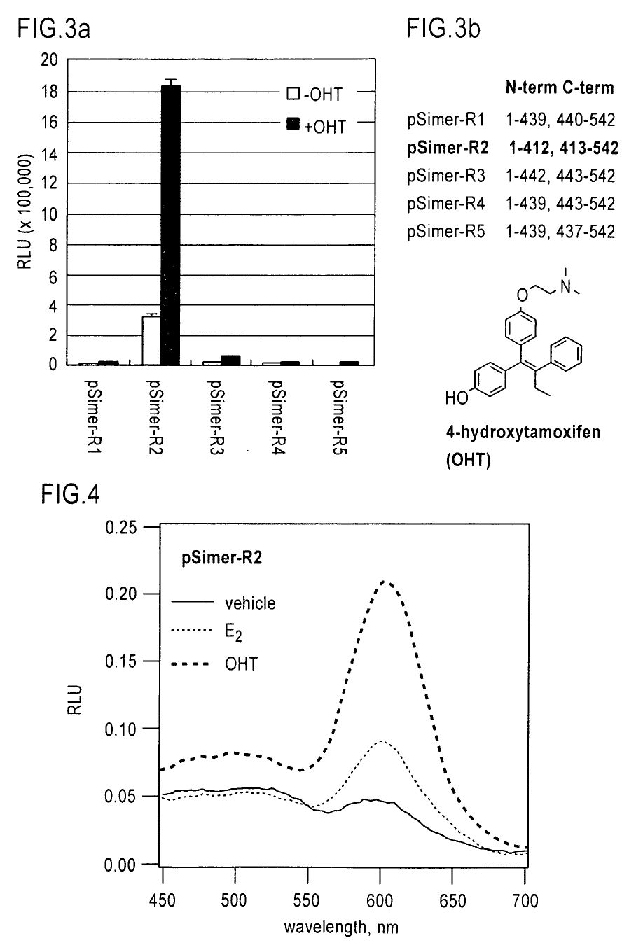

As shown in the above (Example 2), plasmids of pSimer-R1 through —R5 were transfected into COS-7 cells respectively, incubated for 16 hours, and allowed to sufficiently express the bioluminescent probes. Intensity of luminescence emitted by 20 minutes-stimulation was measured by luminometer, provided OHT (4-hydroxytamoxifen), which is a commercially available anticancer drug, is present and absent. As a result, it was observed that cells carrying pSimer-R2 showed the strongest luminescence intencsity in the presence of OHT (FIG. 3).

This result demonstrates that the optimal dissection point of CBLuc-Red is that of pSimer-R2 (between 412 and 413 AA).

Further, measurement of the light spectrum from cells carrying pSimer-R2 with all wavelengths showed that the wavelength of the luminescence was about 610 nm (FIG. 4).

### Example 4

### Measurement of Ligand Sensitivity of Various Cells Carrying pSimer-R2

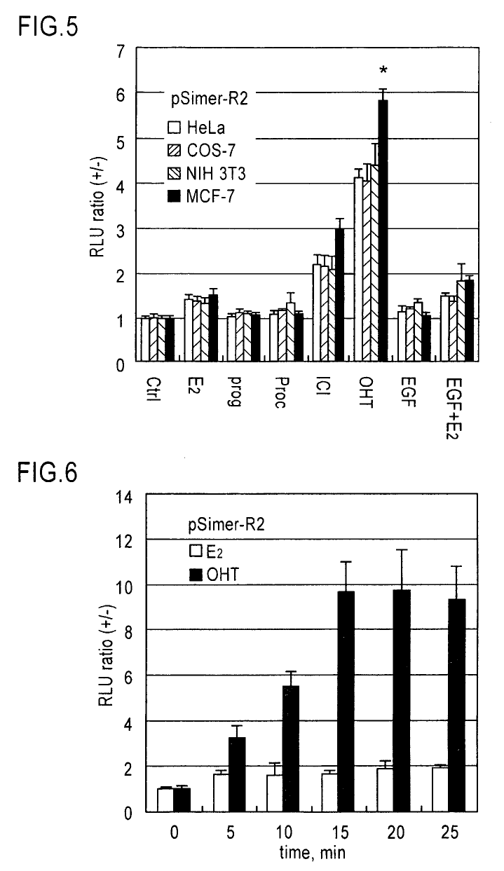

To examine ligand sensitivity of probe in various cell lines, pSimer-R2 was introduced into HeLa cell derived from human cervical cancer, COS-7 cells derived from African Green Monkey's kidney, NIH 3T3 cells derived from murine fibroblast, and MCF-7 cells derived from human breast cancer. Then each cell line was stimulated for 20 minutes with estrogen antagonist (E₂), progesterone (prog), procymidone (proc; fungicide), ICI 182780 (ICI: estrogen antagonist; anti-breastcancer drug), 4-tamoxifen (OHT:4-hydroxytamoxifen; anticancer drug), and epidermal growth factor (EGF), and increase in luminescence intensity was measured by a luminometer (FIG. 5).

As a result, each cell line showed remarkable luminescence intensity especially against stimulation by commercially available anticancer drug, OHT, and anti-breast cancer drug antagonist, ICI.

### Example 5

### Time-Dependent Change of Luminescence Intensity in Cell Carrying pSimer-R2

Change in luminescence intensity depending on ligand stimulation time was observed in COS-7 cells with pSimer-R2 (FIG. 6). The peak luminescence intensity was observed after 15 minutes from OHT stimulation. On the other hand, in simulation with E₂, notable increase in luminescence intensity was not observed.

### Example 6

### Verification of Phosphorylation of ER LBD by Point Mutation

In Example 5, increase in intensity of ligand-dependent luminescence in a cell line carrying pSimer-R2 was caused by phosphorylation of ER LBD by the ligand, it was necessary to clarify whether binding between ER LBD and Src SH2 (phosphorylation recognition domain) was caused by the phosphorylation. Therefore, to verify whether the luminescence change was actually caused by phosphorylation of ER LBD, a negative control probe, pSimer-R2m, was created with deficient phosphorylation site (amino acid at position 537) in ER LBD.

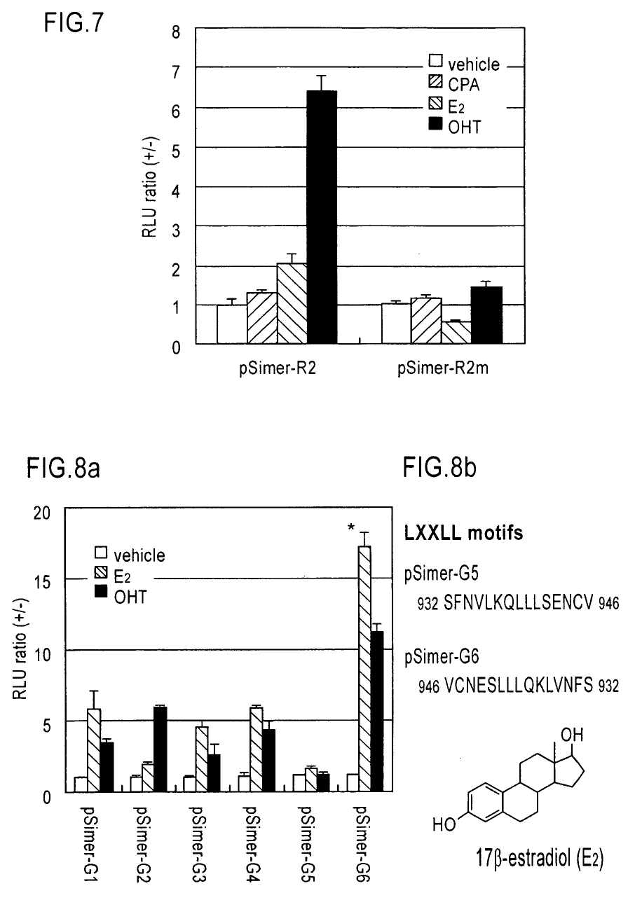

Subsequently, COS-7 cells carrying pSimer-R2 or pSimer-R2m respectively were prepared, and degrees of increase in luminescence intensity by agonists (E₂s) and antagonists (CPAs) were measured. As a result, only in the cells carrying pSimer-R2, ligand-dependent increase in luminescence intensity was observed. On the other hand, ligand sensitivity was not observed in the cells carrying pSimer-R2m (FIG. 7).

Therefore, it can be concluded that ER LBD was phosphorylated by a ligand like OHT, thereby binding Src SH2, as a result, luminescence intensity was increased.

In other words, in the above Example 4, luminescence with strong intensity of red wavelength observed by stimulation with a commercially available anticancer drug, OHT, and antagonist, ICI, reveals that OHT and ICI have an activity of phosphorylating estrogen receptor (ER) and imparting binding ability with phosphorylation recognition domain (SH2) of Src. It also shows that such action in the bioluminescent probe of pSimer-R2 can be applied for screening of substances with antagonistic activity and other anticancer property.

### Example 7

### Determination of Optimal LXXLL Motif Capable of Binding ER LBD

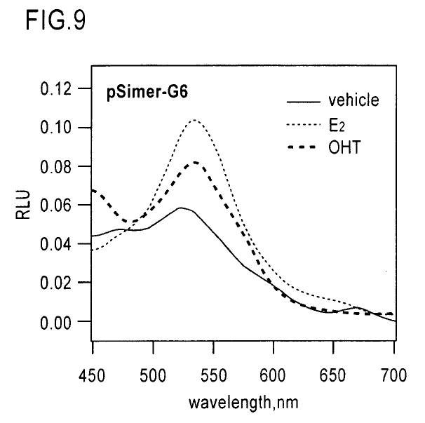

As shown in the above (Table 1), six kinds of LXXLL motifs as those can be applied to each of pSimer-G backbones were prepared, and introduced into pSimer-G. Ligand sensitivity of COS-7 cells carrying either one of the six kinds of plasmids was measured (FIG. 8). Concretely, COS-7 cells were stimulated with E₂ (17β-estradiol: estrogen agonist) or OHT (estrogen antagonist) for 20 minutes, and strongest luminescence intensity ratio relative to control (S/N ratio) was obtained from the cells carrying pSimer-G6. Next, spectrum from the cells carrying pSimer-G6 with all wavelengths was measured, provided ligand was present and absent (FIG. 9). The result shows that luminescence intensity increased depending on estrogen compared to the case of absence of ligand (0.1% DMSO; vehicle), and green light of 540 nm was emitted.

This shows that when a ligand with agonistic activity binds estrogen receptor (ER), conformational change that allows binding a region containing a LXXLL motif occurs, and also shows such action in the pSimer-G series bioluminescent probe is applicable to screening of agonistic substances.

### Example 8

### Measurement of Ligand-Concentration Dependency of Luminescence Intensity in COS-7 Cells Carrying pSimer-G6

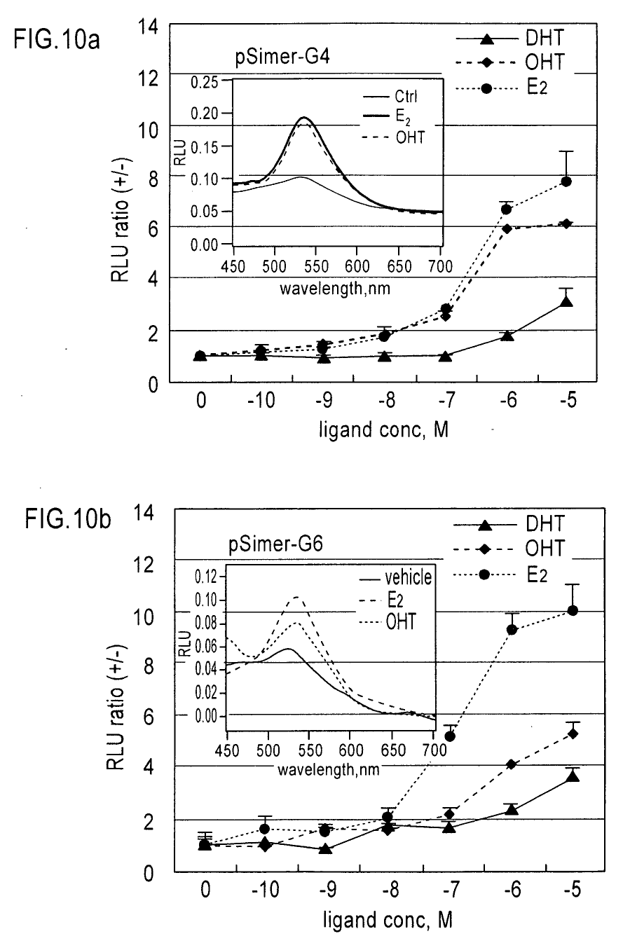

After introducing pSimer-G4 and pSimer-G6 respectively into a cell line in a similar manner as shown above (Example 7), ligand concentration dependency of luminescence intensity in the cell line was examined (FIG. 10). In both cells carrying pSimer-G4 and G6, the luminescence intensity increased from about 10⁻⁸ M and peaked at about 10⁻⁵ M when they were stimulated by E₂. Also the similar results were showed when the cells were stimulated by OHT, except that the concentration at which luminescence intensity started increasing was 10⁻⁶ M OHT in cells carrying pSimer-G6. As to ligand selectivity, selectivity to E₂ was higher than OHT in both cells carrying pSimer-G4 and G6, in particular selectivity to E₂ is very high in the cells carrying pSimer-G6. On the other hand, every cell showed generally low luminescence intensity by stimulation with DHT (androgen agonist).

As a result, pSimer-G6 is the most convincing candidate for a green luminescent probe, and shows good selectivity particularly to agonist (E₂).

### Example 9

### Observation of Ligand Response of COS-7 Cells Cotransfected with Optimal Plasmids in pSimer-R and pSimer-G Serieses (FIG. 1, No. 1 and No. 2)

(9-1) Ligand Response of Cell Co-Expressing pSimer-R2 and pSimer-G4

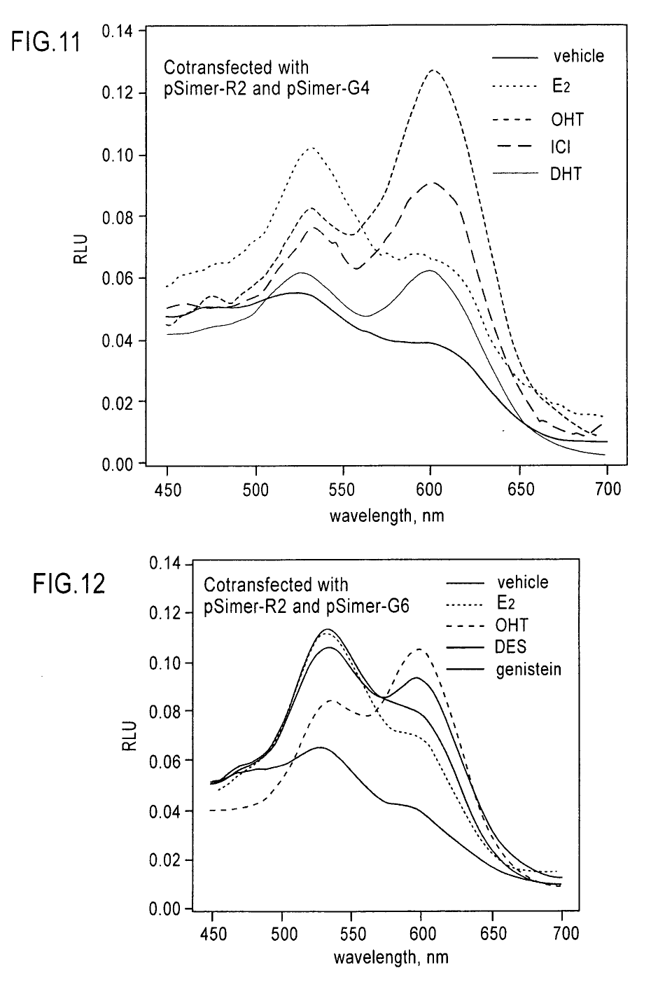

A COS-7 cells carrying both pSimer-R2 and pSimer-G4 were simulated with ligands for 20 minutes, and spectrum of luminescence emitted as a result with all wavelengths was observed (FIG. 11). From that spectrum, two dominant optical densities of red (610 nm) and green (540 nm) were observed. This means that pSimer-R2 and pSimer-G4 were expressed in the same cell, and non-genomic signal transduction (red: involved in carcinogenesis or anticancer) and genomic signal transduction (green; controlling female-sex related phenomena) are respectively activated. These results mean development of sensor cells capable of identifying multiple bioactivities of a ligand in the same cells by colors with different wavelengths. The term genomic signal used herein refers to an intracellular signal involved in conventional protein expression in the nucleus. On the other hand, the term non-genomic signal means an intracellular signal not accompanying conventional protein expression. Typically, this is the general classification of intracellular signal transductions.

Further, from ratio of luminescence intensities between red signal (610 nm) and green signal (540 nm) induced by a ligand, it is possible to show numerically whether the measured ligand tends to genomic activity (also referred to as agonistic activity herein) or nongenomic activity (also referred to as antagonistic activity herein). To evaluate ligand response by COS-7 cells cotransfected with pSimer-R2 and pSimer-G4 in scores, a ratio of intensity variations of luminescence at 540 nm and 610 nm by the ligand is indicated, and this ratio is named “estrogenicity score.” This calculational method is as shown in [Table 2], and [Table 2] also shows scores of the respective ligands. According to this calculational method, E₂ has a score of 1.62, and estrogen antagonist OHT shows a score of 0.62.

TABLE 2
Estrogenicity score of each ligand in cells
contransfected with pSimer-R2 and pSimer-S4

| | | Cross-talk | Endcrine |
|---|---|---|---|
| Agonist | Antagonist | Agonist | Disrupter |
| E₂ | OHT | DHT | PCB |
| 1.62 | 0.62 | 0.87 | 0.86 |
| | genisteine | T | flutamide |
| | 0.84 | 1.07 | 0.97 |
| | ICI | | CPA |
| | 0.86 | | 1.19 |

Formula:
Estrogenicity score = [variation of luminescence intensity at 540 nm]/[variation of luminescence intensity at 610 nm]
Abbreviations: E₂, 17β-estradiol; DES, diethylstilbestrol; OHT, 4-hydroxytamoxifene; ICI, ICI182780; DHT, 5α-dihydrotestosterone; T, testosterone; PCB, polychlorinated biphenyls; CPA, cyproterone acetate.

(9-2) Ligand Response of Cells that is Cotransfected with pSimer-R2 and pSimer-G6

As a related experiment, COS-7 cells in which pSimer-R2 and pSimer-G6 were co-expressed was stimulated for 20 minutes with ligands, and spectrum of the resultant luminescence with all wavelengths was observed (FIG. 12). Similarly shown in FIG. 11, bands of luminescence intensity were observed around 540 nm and 610 nm. According to the same method for calculating estrogenicity score, agonist E₂ had a score of 1.8, and estrogen antagonist OHT showed a score of 0.38. For other ligands, respective scores are shown in [Table 3].

By indicating with such scores, antagonistic/agonistic activity of each ligand can be seen in scores and objectively.

TABLE 3
Estrogenicity score of each ligand in cells co-expressing
pSimer-R2 and pSimer-G6 plasmids

| | | Cross-talk | Endcrine |
|---|---|---|---|
| Agonist | Antagonist | Agonist | Disrupter |
| E₂ | OHT | DHT | PCB |
| 1.80 | 0.38 | 0.87 | 0.86 |
| DES | genisteine | T | flutamide |
| 1.27 | 0.82 | 1.07 | 0.97 |
| estrone | ICI | progesterone | CPA |
| 0.95 | 1.08 | 1.06 | 1.19 |
| | | cortisol | RU486 |
| | | 0.68 | 1.33 |

Formula:
Estrogenicity score = [variation of luminescence intensity at 540 nm]/[variation of luminescence intensity at 610 nm]
Abbreviations: E2, 17β-estradiol; DES, diethylstilbestrol; OHT, 4-hydroxytamoxifene; ICI, ICI182780; DHT, 5α-dihydrotestosterone; T, testosterone; PCB, polychlorinated biphenyls; CPA, cyproterone acetate.

### Example 10

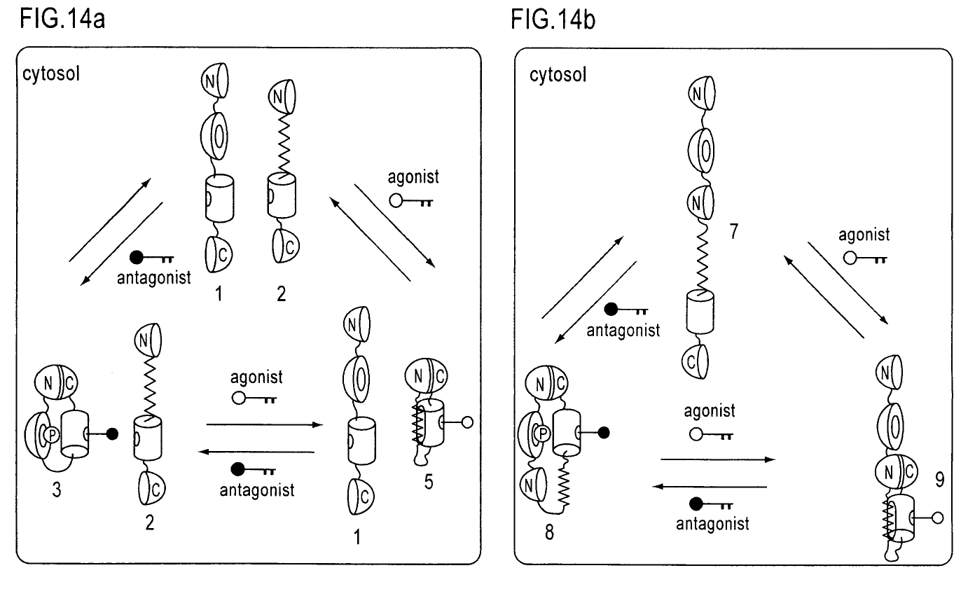

### Measurement of Ligand Dependency of COS-7 Cells Carrying pSimer-RG Series Plasmid (FIG. 1, No. 7; FIG. 14, No. 7)

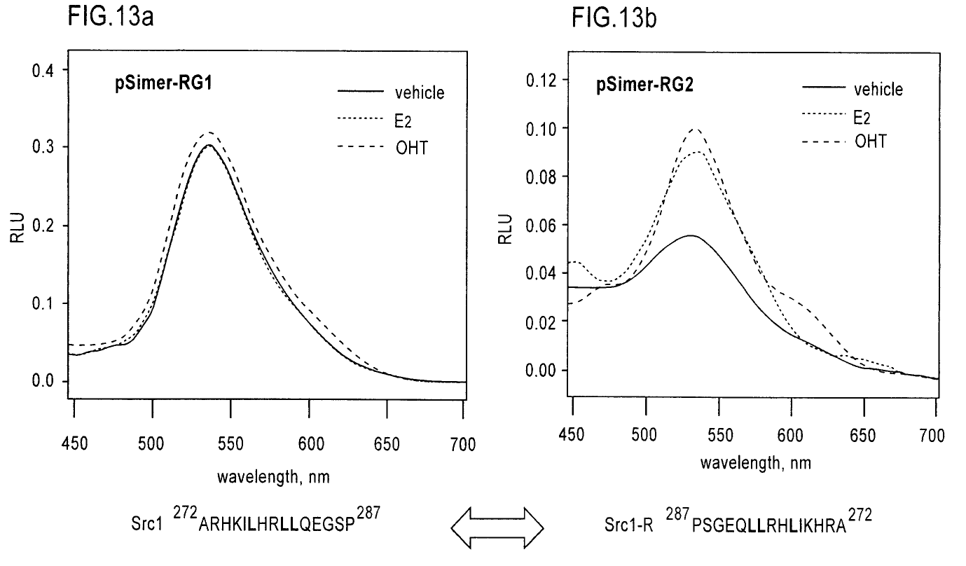

Spectrum of ligand dependent luminescence with all wavelengths of COS-7 cells respectively carrying pSimer-RG1 or pSimer-RG2 were observed (FIG. 13). In FIG. 13, a and b are spectra of COS-7 cells transfected with pSimer-RG1 or pSimer-RG2 respectively.

Here, pSimer-RG2 was constructed by assembling a cDNA sequence encoding a usual LXXLL motif; however, pSimer-RG1 was designed so that the cDNA fragment was connected in reverse order to make “LLXXL” sequence allocated in the position corresponding to “LXXLL”.

As a result, the cells carrying pSimer-RG2 to which LXXLL motif is properly connected exhibited different spectrum respectively when stimulated for 20 minutes with E₂, OHT, or vehicle. This result demonstrates that estrogen activity and anticancer activity of a ligand can be simultaneously distinguished by spectrum pattern with the probe having a proper LXXLL motif. In other words, it is demonstrated that by conformational change in a single molecule, degree of multiple bioactivities of a ligand can be displayed in short time as multicolor luminescence signals.

On the other hand, as to the ligand dependent luminescence spectrum in the cells carrying pSimer-RG1 with improper LXXLL motif, specific variation in spectra was not exhibited by stimulation of any of E₂, OHT, and vehicle (0.1% DMSO). In this case, high background intensity of green luminescence is already exhibited at the time of vehicle stimulation, and it can be considered that equilibrium is predominantly shifted to the structure of No. 9 in the probe molecule equilibrium chart of FIG. 14b.

Since the single-molecule-format multicolor bioluminescent probe expressed by pSimer-RG2 can clearly distinguish signal spectrum from the background spectrum, and different spectrum patterns are exhibited for different ligands, it is possible to qualitatively and quantitatively analyze agonistic and/or antagonistic activity of a ligand by respective luminescence spectrum patterns just like a fingerprint as shown in FIG. 13b.

1. A multicolor luminescent probe set that emits two-dimensional luminescence signals of wavelength and intensity in response to a property and level of bioactivity of a ligand, comprising:
(1) a single-molecule-format bioluminescent probe that emits the light of the first wavelength, wherein a fusion protein comprising the first molecular recognition domain and a ligand recognition protein which becomes capable of binding said first molecular recognition domain due to the first conformational change sandwiched between the split N-terminal fragment (N-LE-1) and C-terminal fragment (C-LE-1) of the first Lighting Enzyme (LE-1); and
(2) a singl-moleucle-format bioluminescent probe that emits the light of the second wavelength, wherein a fusion protein comprising the second molecular recognition domain and said ligand recognition protein which becomes capable of binding said second molecular recognition domain due to the second conformational change sandwiched between the split N-terminal fragment (N-LE-2) and C-terminal fragment (C-LE-2) of the second Lighting Enzyme (LE-2).

2. The multicolor luminescent probe set according to claim 1, wherein the first Lighting Enzyme is a luciferase emitting green luminescence (Luc-Green) and the wavelength of the first luminescence signal is within green range, and the second Lighting Enzyme is a luciferase emitting red luminescence (Luc-Red) and the wavelength of the second luminescent signal is within red range.

3. The multicolor luminescent probe set according to claim 1 or 2, further comprising a single-molecule-format bioluminescent probe that emits light of the third wavelength, wherein a fusion protein comprising the third molecular recognition domain and the ligand recognition protein either the same or different from said ligand recognition protein which becomes capable of binding said third molecular recognition domain due to the third conformational change sandwiched between the split N-terminal fragment (N-LE-3) and C-terminal fragment (C-LE-3) of the third Lighting Enzyme (LE-3).

4. The multi color luminescent probe set according to claim 3, wherein the third Lighting Enzyme is a luciferase emitting yellow luminescence (Luc-Yellow) and the wavelength of the third luminescence signal is within yellow range.

5. The multicolor luminescent probe set according to claim 1, wherein the ligand recognition protein is the nuclear receptors (NRs), cytokine receptors, or various protein kinases that recognizes a hormone, chemical substance or protein for signal transduction as a ligand.

6. The multicolor luminescent probe set according to claim 5, wherein the ligand recognition protein is the nuclear receptors (NRs) selected from the group consisting of estrogen receptors (ERs), glucocorticoid receptors (GRs), androgen receptors (ARs), and progesterone receptors (PRs).

7. The multicolor luminescent probe set according to claim 5 or 6, wherein the first molecular recognition domain is derived from a coactivator, and the second molecular recognition domain is the protein phosphorylation recognition domain when the ligand recognition protein is the nuclear receptors (NRs).

8. The multicolor luminescent probe set according to claim 7, wherein the molecular recognition domain derived from a coactivator is a molecular recognition domain comprising LXXLL motif, and the protein phosphorylation recognition domain is SH2 domain derived from kinase.

9. The multicolor luminescent probe set according to claim 1, further comprising a single-molecule-format bioluminescent probe that emits light of the n-th wavelength, wherein a fusion protein comprising n-th molecular recognition domain and the ligand recognition protein either the same or different from said ligand recognition protein which becomes capable of binding to said n-th molecular recognition domain due to the conformational change sandwiched between the split N-terminal fragment (N-LE-n) and C-terminal fragment (C-LE-n) of the n-th Lighting Enzyme (LE-n), provided that n is a natural number of 3 or larger.

10. A single-molecule-format multicolor bioluminescent probe that emits two-dimensional luminescence signals of wavelength and intensity in response to property and level of bioactivity of a ligand, wherein a fusion protein comprising the first molecular recognition domain and a ligand recognition protein which becomes capable of binding said first molecular recognition domain due to the first conformational change sandwiched between the split N-terminal fragment (N-LE-1) and C-terminal fragment (C-LE-1) of the first Lighting Enzyme (LE-1); wherein the second molecular recognition domain bound by said ligand recognition protein which acquired a binding capability due to the second conformational change is connected further to the N-terminal side of the N-LE-1, and further to the N-terminal side thereof the split N-terminal fragment (N-LE-2) of the second Lighting Enzyme (LE-2) is connected; wherein self-complementation between N-LE-1 and C-LE-1 takes place when the first molecular recognition domain is bound by the ligand recognition protein due to the first conformational change as a result of binding of the ligand by the ligand recognition protein, and triggers emission of the luminescence signal of the first wavelength; in like wise, the second molecular recognition domain is bound by the ligand recognition protein due to the second conformational change, followed by the self-complementation between N-LE-2 and C-LE-1 takes place, and thereby triggers emission of the luminescence signal of the second wavelength.

11. The single-molecule-format bioluminescent probe according to claim 10, wherein the first Lighting Enzyme is luciferase emitting green light (Luc-Green) and wavelength of the first luminescence signal is within green range, and the second Lighting Enzyme is luciferase emitting red light (Luc-Red) and wavelength of the second luminescence signal is within red range.

12. The single-molecule-format bioluminescent probe according to claim 10 or 11, wherein the ligand recognition protein is the nuclear receptors (NRs), cytokine receptors, or various protein kinases.

13. The single-molecule-format multicolor luminescent probe according to claim 12, wherein the ligand recognition protein is the nuclear receptors (NRs) selected from the group consisting of estrogen receptors (ERs), glucocorticoid receptors (GRs), androgen receptors (ARs), and progesterone receptors (PRs).

14. The single-molecule-format multicolor bioluminescent probe according to claim 10, wherein the first molecular recognition domain is derived from a coactivator, and the second molecular recognition domain is the protein phosphorylation recognition domain.

15. The single-molecule-format multicolor bioluminescent probe according to claim 14, wherein the molecular recognition domain derived from a coactivator is a molecular recognition domain comprising LXXLL motif, and the phosphorylation recognition domain is SH2 domain derived from a kinase.

16. A set of nucleic acids encoding multicolor bioluminescent probe set that emits two-dimensional luminescent signals of wavelength and intensity in response to property and bioactivity level of a ligand; and capable of expressing the multicolor luminescent probe set of claim 1 in a living cell line comprising:
(1) the nucleic acids encoding a single-molecule-format bioluminescent probe that emits light of the first wavelength, wherein the nucleic acids encoding a fusion protein comprising the first molecular recognition domain and a ligand recognition protein which becomes capable of binding said first molecular recognition domain due to the first conformational change sandwiched between the nucleic acids encoding the split N-terminal fragment (N-LE-1) and C-terminal fragment (C-LE-1) of the first Lighting Enzyme (LE-1); and
(2) the nucleic acids encoding a singl-moleucle-format bioluminescent probe that emits the light of the second wavelength, wherein the nucleic acids encoding a fusion protein comprising the second molecular recognition domain and said ligand recognition protein which becomes capable of binding said second molecular recognition domain due to the second conformational change sandwiched between the nucleic acids encoding the split N-terminal fragment (N-LE-2) and C-terminal fragment (C-LE-2) of the second Lighting Enzyme (LE-2).

17. An expression vector wherein a set of nucleic acids encoding the multicolor luminescent probe set according to claim 16 is inserted or a set of expression vectors wherein each of the nucleic acids are inserted, and said single expression vector or said expression vector set which is capable of expressing a multicolor luminescent probe set that emits two-dimensional luminescent signals of wavelength and intensity in response to property and bioactivity level of a ligand.

18. A transfected living cell line, wherein the expression vector or the vector set according to claim 17 is inserted, thereby expressing a multicolor luminescent probe that emits two-dimensional luminescent signals of wavelength and intensity in response to property and bioactivity level of a ligand.

19. Nucleic acids encoding a single-molecule-format multicolor bioluminescent probe that emits two-dimensional luminescent signal of wavelength and intensity in response to property or bioactivity level of a ligand, wherein the nucleic acids encoding the single-molecule-format multicolor bioluminescent probe according to claim 10, wherein the nucleic acids encoding a fusion protein of the first molecular recognition domain and the ligand recognition protein sandwiched between nucleic acids encoding split N- and C-terminal fragment of the first Lighting Enzyme (LE-1), wherein the nucleic acids encoding the second molecular recognition domain are connected further to the N-terminal side of the nucleic acids encoding N-LE-1, and further to the N-terminal side thereof are connected the nucleic acids encoding the split N-terminal fragment (N-LE-2) of the second Lighting Enzyme (LE-2), wherein self-complementation between N-LE-1 and C-LE-1 takes place when the first molecular recognition domain is bound by the ligand recognition protein as a result of the first conformational change, triggers the emission of the luminescent signal of the first wavelength; whereas self-complementation between N-LE-2 and C-LE-1 takes place when the second molecular recognition domain is bound by the ligand recognition protein as a result of the second conformational change, triggers the emission of the luminescent signal of the second wavelength.

20. An expression vector comprising the nucleic acids encoding a single-molecule-format multicolor bioluminescent probe according to claim 19, wherein the expression vector is capable of expressing a single-molecule-format multicolor bioluminescent probe that emits two-dimensional luminescent signals of wavelength and intensity in response to property and bioactivity level of a ligand in a living cell line.

21. A living cell line expressing a single-molecule-format multicolor bioluminescent probe that emits two-dimensional luminescent signal with wavelength and intensity in response to property and bioactivity level of a ligand, wherein the living cell line is transfected with the expression vector according to claim 20, comprising the nucleic acids encoding a single-molecule-format multicolor bioluminescent probe.

22. A method for qualitative and quantitative evaluation of the activity of test substance as a ligand for the subject protein, wherein the method comprises the step of stimulating a multicolor luminescent probe set expressed in a living cell line according to claim 18 with the test substance; followed by measuring the wavelength and intensity of luminescence; and thereby analyzing the test substance for property and level of bioactivity.

23. A method for determining antagonist/agonist activity of a test substance, wherein the method comprises the steps of stimulating a multicolor luminescent probe set expressed in a living cell according to claim 18 with the test substance; followed by evaluating each lighting enzyme with the changes in luminescence intensity ratios before and after the stimulation.

24. A method of screening antagonist and/or agonist against the subject ligand recognition protein, wherein the method comprises the steps of stimulating the multicolor luminescent probe set expressed in a living cell according to claim 18 with the test substance; followed by measuring wavelength and intensity of the luminescence.

25. A kit for qualitative and quantitative evaluation of the activity of the test substance as a ligand for the subject protein, comprising the living cell line which expresses the multicolor luminescent probe set according to claim 18.

26. A kit for screening antagonist and/or agonist against the ligand recognition protein, comprising the living cell lines which expresses the multicolor luminescent probe set according to claim 18.

27. A kit for qualitative and quantitative analysis for the ligand activity or a kit for screening antagonist and/or agonist against the ligand recognition protein, wherein combined a Lighting Enzyme substrate used for each luminescent probe with the single expression vector or the set of expression vectors according to claim 17 which can express the multicolor luminescent probe set in a living cell.

28. A kit for qualitative and quantitative analysis for ligand activity or a kit for screening antagonist and/or agonist against a ligand recognition protein, wherein combined a Lighting Enzyme substrate used for each luminescent probe with the multicolor luminescent probe set according to any of claim 1.

29. A method for qualitative and quantitative evaluation of the activity of test substance as a ligand for the subject protein, wherein the method comprises the step of stimulating a single-molecule-format multicolor bioluminescent probe expressed in a living cell according to claim 21 respectively with the test substance; followed by measuring the wavelength and intensity of luminescence; and thereby analyzing the test substance for property and level of bioactivity.

30. A method for determining antagonist/agonist activity of a test substance, wherein the method comprises the steps of stimulating a single-molecule-format multicolor bioluminescent probe expressed in a living cell according to claim 21 with the test substance; followed by evaluating each lighting enzyme with the changes in luminescence intensity ratios before and after the stimulation.

31. A method of screening antagonist and/or agonist against the subject ligand recognition protein, wherein the method comprises the steps of stimulating the single-molecule-format multicolor bioluminescent probe expressed in a living cell according to claim 21 with the test substance; followed by measuring wavelength and intensity of the luminescence.

32. A kit for qualitative and quantitative evaluation of the activity of the test substance as a ligand for the subject protein, comprising the living cell line which expresses the single-molecule-format multicolor bioluminescent probe according to claim 21.

33. A kit for screening antagonist and/or agonist against the ligand recognition protein, comprising the living cell line which expresses the single-molecule-format multicolor bioluminescent probe according to claim 21.

34. A kit for qualitative and quantitative analysis for the ligand activity or a kit for screening antagonist and/or agonist against the ligand recognition protein, wherein combined a Lighting Enzyme substrate used for each luminescent probe with the expression vector according to claim 20 which comprises the nucleic acids encoding a single-molecule-format multicolor luminescent probe.

35. A kit for qualitative and quantitative analysis for ligand activity or a kit for screening antagonist and/or agonist against a ligand recognition protein, wherein combined a Lighting Enzyme substrate used for each luminescent probe with the single-molecule-format multicolor luminescent probe according to claim 10.


SEQUENCE LISTING (10 sequences)

SEQ ID NO: 1 - protein, 15 aa, Artificial
  pSimer-G1(Referred from Human steroid receptor coactivator Src-1)
ARHKILHRLLQEGSP

SEQ ID NO: 2 - protein, 15 aa, Artificial
  pSimer-G2(Referred from Human steroid receptor coactivator Src-1)
PSGEQLLRHLIKHRA

SEQ ID NO: 3 - protein, 15 aa, Artificial
  pSimer-G3(Referred from Human coactivator Rip140)
HQKVTLLQLLLGHKN

SEQ ID NO: 4 - protein, 15 aa, Artificial
  pSimer-G4 (Referred from Human coactivator Rip140)
NKHGLLLQLLTVKQH

SEQ ID NO: 5 - protein, 15 aa, Artificial
  pSimer-G5(Referred from Human coactivator Rip140)
SFNVLKQLLLSENCV

SEQ ID NO: 6 - protein, 15 aa, Artificial
  pSimer-G6(Referred from Human coactivator Rip140)
VCNESLLLQKLVNFS

SEQ ID NO: 7 - protein, 17 aa, Artificial
  Signal peptide(Referred from N-terminal sequence information of Gaussia luciferase)
MGVKVLFALICIAVAEA

SEQ ID NO: 8 - protein, 6 aa, Artificial
  His-tag
HHHHHH

SEQ ID NO: 9 - protein, 3 aa, Artificial
  GS linker, consisted of 3 amino acids
GGG

SEQ ID NO: 10 - protein, 10 aa, Artificial
  GS linker, consisted of 10 amino acids
GGGGRNGGGA